(19) United States
(12) Patent Application Publication
(10) Pub. No.: US 2020/0030445 A1
(43) Pub. Date: Jan. 30, 2020

(54) STABLE FORMULATIONS OF HUMANIZED ANTI-TAU ANTIBODY

(71) Applicant: C2N Diagnostics, LLC, St. Louis, MO (US)

(72) Inventors: Elizabeth John, Richland, WA (US); Philip Verghese, St. Louis, MO (US); Yan Hu, St. Louis, MO (US); Matthias Kron, Ulm (DE); Katja Zipfel, Laupheim (DE); Alexander Faude, Burgrieden (DE)

(21) Appl. No.: 15/735,728

(22) PCT Filed: Jun. 13, 2016
(86) PCT No.: PCT/US2016/037236

§ 371 Date: Dec. 12, 2017

Related U.S. Application Data
(60) Provisional application No. 62/175,154, filed on Jun. 12, 2015.

(51) Int. Cl. A61K 39/395; C07K 16/18; A61K 47/26; A61K 47/10; A61K 47/22; A61K 47/14

(52) U.S. Cl. CPC A61K 39/39591; C07K 16/18; A61K 47/26; C07K 2317/24; A61K 47/22; A61K 47/14; A61K 47/10

(57) ABSTRACT

Provided herein are stable compositions containing anti-tau antibodies. In particular, compositions containing C2N-8E12 anti-tau antibody and a buffering system are provided herein.

### BACKGROUND OF THE INVENTION

### Field of the Invention

The invention relates generally to stable anti-tau antibody formulations and specifically to a stable formulation of the C2N-8E12 anti-tau antibody to improve sensitivity to thermal and mechanical stress.

### Background of the Invention

Alzheimer's disease (AD) is a common chronic progressive neurodegenerative disease in which there is an irreversible loss of cognitive and behavioral functions. The disease can persevere for over 10 years, advancing from mild symptoms to extremely severe manifestations. AD is said to afflict approximately 10% of the population over the age of 65 and more than 30% of the population over the age of 80.

Pathologically, Alzheimer's disease presents as extracellular amyloid plaques and intracellular neurofibrillary tangles. The neurofibrillary tangles are composed, e.g., of the microtubule-binding protein tau, which is assembled into paired helical and straight filaments. It has been suggested that these entities may be functionally linked, although the mechanisms by which amyloid deposition promotes pathological tau filament assembly is not clear.

The common denominator of intracellular neurofibrillary structures (neurofibrillary tangles, dystrophic neurites, and neurophil threads) is paired helical filaments (PHFs). The major protein subunit of the PHFs is microtubule associated protein tau in abnormally hyperphosphorylated form. Neurons with neurofibrillary changes degenerate, and the degree of this degeneration directly correlates with the degree of dementia in the affected individuals.

A number of neurological diseases are known to have filamentous cellular inclusions containing microtubule associated protein tau, e.g., Alzheimer's disease (AD), progressive supranuclear palsy (PSP), corticobasal degeneration (CBD), Pick's disease (PiD) and a group of related disorders collectively termed frontotemporal dementia with Parkinsonism linked to chromosome 17 (FTDP-17), amyotropic lateral sclerosis (ALS), Creutzfeldt-Jakob disease (CJD), dementia pugilistica (DP), Gerstmann-Straussler-Scheinker disease (GSSD), Lewy body disease and Huntington disease. Although the etiology, clinical symptoms, pathologic findings and the biochemical composition of inclusions in these diseases are different, there is emerging evidence suggesting that the mechanisms involved in aggregation of normal cellular proteins to form various filamentous inclusions are comparable. It is believed, that an initial alteration in conformation of microtubule associated protein tau, that initiates generation of nuclei or seeds for filament assembly, is one of the key features. This process can be influenced by the posttranslational modification of normal proteins, by mutation or deletion of certain genes and by factors that bind normal proteins and thus alter their conformation.

Strong experimental evidence and biological rationale exists to support the tau immunotherapy strategy as a way to counter tau pathology in neurodegeneration. First, tau is normally a highly soluble, natively unfolded, and intracellular protein, so an extracellular antibody is unlikely to affect the normal functions of tau. Second, the burden of tau pathology correlates with progressive neuronal dysfunction, synaptic loss, and functional decline in humans and transgenic mouse models of tauopathy. Third, under pathological conditions, tau becomes misfolded and aggregates into intraneuronal neurofibrillary tangles (NFTs) composed of pathological tau fibrils. In human tauopathies, this pathology progresses from one brain region to another in disease-specific patterns. Experimental data suggests that tau aggregates can spread from cell to cell to induce further tau aggregation and spreading of tau pathology in brain. This data suggests that aggregates produced in one cell are released into the extracellular space and can promote aggregation in neighboring or connected cells. Finally, it has been demonstrated that anti-tau antibodies can prevent or slow the progression of tau pathology in the brain of mice that carry mutated human form of tau.

### SUMMARY OF THE INVENTION

The present invention is based on the discovery of stable formulations of the C2N-8E12 anti-tau antibody at a concentration of 20 mg/mL, aproduct monomer content of >95% and a pH in the physiological range.

Provided herein are compositions including (i) a humanized antibody which specifically binds TAU comprising a heavy chain variable (VH) region and a light chain variable (VL) region, having an amino acid sequence as set forth in FIGS. 1 and 2; and (ii) a buffering system that optionally contains an excipient and/or optionally contains a surfactant.

In certain embodiments, the humanized antibody is C2N-8E12. In one embodiment, the concentration of the humanized antibody in the buffering system is about 20 mg/mL. In other embodiments, the excipient is saccharose, sorbitol or glycine. In yet other embodiments, the surfactant is Tween20.

By way of example, the buffering system of the compositions provided herein may include: (a) 50 mM L-Histidine/HCl, 8.6% Saccharose at pH 7.0; (b) 50 mM L-Histidine/HCl, 8.6% Saccharose at pH 6.5; (c) 50 mM L-Histidine/HCl, 8.6% Saccharose at pH 6.0; (d) 50 mM L-Histidine/HCl, 5.3% Sorbitol at pH 7.0; (e) 30 mM Na-citrate, 9.2% Saccharose at pH 6.0; (f) 30 mM Na-acetate, 9.2% Saccharose at pH 5.5; (g) 30 mM Na-acetate, 2.0% Glycine at pH 5.5; or (h) 30 mM Glycyl-Glycine, 9.2% Saccharose at pH 7.0.

In one embodiment, the buffering system comprises 50 mM Histidine, and 8.6% Saccharose and may optionally contain a surfactant, such as Tween20 or polysorbate 20.

In another embodiment, the buffering system is 50 mM Histidine and 8.6% Saccharose. In one embodiment, polysorbate 20 is present in an amount of about 0.02%.

In certain aspects, the concentration of the humanized antibody in the compositions provided herein is about 20 mg/mL. In one embodiment, the buffering system comprises about 50 mM Histidine, about 8.6% Saccharose and about 0.02% Tween 20 at about pH 6.0.

Also provided herein are compositions including (i) a humanized antibody which specifically binds TAU comprising a heavy chain variable (VH) region as set forth in FIGS. 1 and 2 and a light chain variable (VL) region as set forth in FIGS. 1 and 2; and (ii) a buffering system comprising histidine, citrate, or glyclglycine, and that optionally contains an excipient and/or optionally contains a surfactant.

In certain aspects, the buffering system comprises histidine and an excipient selected from saccharose, sorbitol, and combinations thereof. In other aspects, the buffering system comprises citrate and an excipient selected from saccharose, glycine, and combinations thereof. In yet other aspects, the buffering system comprises glycylglycine and the excipient is saccharose. In one embodiment, the buffering system comprises histidine/HCl and does not include sodium. In another embodiment, the anti-tau antibody is present at a concentration of about 20 mg/mL and the composition has a monomer content of about >95%, a pH between about 5 and 6, and is stable for at least three months when stored at about 5° C.

### BRIEF DESCRIPTION OF THE DRAWINGS

FIG. 1 shows the variable region sequences of the murine HJ8.5 (C2N-8E12) antibody as well as the 4 humanized variant sequences for each of the heavy and light chains (4 VH and 4 VL/K sequences). The CDR sequences are highlighted in red. Framework changes from the original mouse sequence are highlighted in blue.

FIG. 2 shows the sequences of the grafted variable and constant region sequences for each of the heavy and light chains (4 VH and 4 VL/K sequences). The variable heavy chain is grafted to the constant heavy chain of human IgG4 containing the S241P hinge stabilizing mutation. The variable light chain is grafted to the constant light chain of human Kappa light chain. This table also lists the theoretical isoelectric point (pI) and molecular weight (Mw).

### DETAILED DESCRIPTION OF THE INVENTION

The following terms, definitions and abbreviations apply. Abbreviations used herein have their conventional meaning within the chemical and biological arts.

In the context of the disclosure, the term “subject” generally refers to living organisms, e.g., prokaryotes and eukaryotes who will receive or who has received treatment described below (e.g., administration of the compositions of the disclosure, and optionally one or more additional therapeutic agents). Examples of subjects include mammals, e.g., humans, dogs, cows, horses, pigs, sheep, goats, cats, mice, rabbits, rats, and transgenic non-human animals. In certain embodiments, the subject is a human.

The disclosure also provides pharmaceutical compositions comprising at least one anti-tau antibody in an amount effective for treating a disorder, and a pharmaceutically acceptable vehicle or diluent. The compositions of the disclosure may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The disclosed pharmaceutical compositions may be administered by any suitable means, for example, parenterally, such as by subcutaneous, intravenous, intramuscular, intracerebroventricular, intrathecal, or intracisternal injection or infusion techniques (e.g., as sterile injectable aqueous solutions); in dosage unit formulations containing non-toxic, pharmaceutically acceptable vehicles or diluents. The present compounds may, for example, be administered by continuous infusion by the use of devices such as subcutaneous implants or continuous infusion pumps. The present compositions may also be administered liposomally.

The term “therapeutically effective amount” means the amount of the antibody or pharmaceutical composition that will elicit the biological or medical response of a tissue, system, animal or human that is being sought by the researcher, veterinarian, medical doctor or other clinician.

By “pharmaceutically acceptable” it is meant the buffer, excipient, or surfactant must be compatible with the other ingredients of the formulation and not deleterious to the subject.

The terms “administration of” and or “administering a” composition or antibody should be understood to mean providing a composition or antibody of the disclosure or pharmaceutical composition to the subject in need of treatment.

A “stable” formulation is one in which the antibody therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker. Inc., New York, N.Y., Pubs. (1991) and Jones, A. Adv. Drug Delivery Rev. 10: 29-90 (1993), for example. Stability can be measured at a selected temperature for a selected time period. Stability can be measured by thermal transition points via thermofluorescence (TF) assays. Preferably, the formulation is stable at room temperature (about 30° C.) or at 40° C. for at least 1 month and/or stable at about 2-8° C. for at least 1 year for at least 2 years. Furthermore, the formulation is preferably stable following freezing (to, e.g., −70° C.) and thawing of the formulation, hereinafter referred to as a “freeze/thaw cycle.”

The term “buffering system” mans a buffered liquid that resists changes in pH by the action of its acid-base conjugate components. The buffer of this invention has a pH in the range from about 4 to about 8; preferably from about 5 to about 7.5; and most preferably has a pH in the range from about 5.5 to about 7. Examples of buffers that will control the pH in this range include acetate (e.g. sodium acetate), succinate (such as sodium succinate), gluconate, histidine, citrate, glycylglycine and other organic acid buffers. In some embodiments, the buffering system comprises one or more of histidine, citrate, or glycylglycine.

In some embodiments, the composition comprises a surfactant. Exemplary surfactants include nonionic surfactants such as polysorbates (e.g. polysorbates 20, 80, etc.) or poloxamers (e.g. poloxamer 188). The amount of surfactants added is such that it reduces aggregation of the formulated antibody and/or minimizes the formation of particulates in the formulation and/or reduces adsorption. In a preferred embodiment of the invention, the formulation includes a surfactant which is a polysorbate. In another preferred embodiment of the invention, the formulation contains the detergent polysorbate 20 or Tween 20. Tween 20 is a term used to describe polyethylene glycol (20) sorbitan monolaurate. In one preferred embodiment, the formulation contains between about 0.1 and about 10 mg/ml of polysorbate 20, more preferably between about 0.5 and about 5 mg/ml. In another preferred embodiment, about 0.1% polysorbate 20 is found in the formulation of the invention.

The pharmaceutical compositions for the administration of the antibodies of this disclosure either alone or in combination with other agents, e.g., chemotherapeutic, may conveniently be presented in dosage unit form and may be prepared by any of the methods well known in the art of pharmacy. In the pharmaceutical composition the active object compound is included in an amount sufficient to produce the desired effect upon the process or condition of diseases. The pharmaceutical compositions containing the active ingredient may be in a form suitable for parenteral use and/or for aqueous solutions or suspensions.

Aqueous solutions and suspensions contain the active materials in solution or admixture with excipients suitable for the manufacture of aqueous suspensions. Suitable excipients include but are not limited to suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents such as a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Suitable excipients also include solubilizers such as polyethylene glycol, for example. The aqueous suspensions may also contain excipients such as one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example coloring agents, may also be present.

The sterile injectable composition may also be a sterile injectable solution or suspension in a parenterally-acceptable diluent or solvent or cosolvent or complexing agent or dispersing agent or excipient or combination thereof, for example 1,3-butane diol, polyethylene glycols, polypropylene glycols, ethanol or other alcohols, povidones, Tweens, sodium dodecyle sulfate, sodium deoxycholate, dimethylacetamide, polysorbates, poloxamers, cyclodextrins, e.g., sulfobutyl ether J-cyclodextrin, lipids, and excipients such as inorganic salts (e.g., sodium chloride), buffering agents (e.g., sodium citrate, sodium phosphate), and sugars (e.g., saccharose and dextrose). Among the acceptable vehicles and solvents that may be employed are water, dextrose solutions, Ringer's solutions and isotonic sodium chloride solution.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intracerebroventricularly, intrathecal, intraventricular, intravenous, intraperitoneal, or intranasal administration. For injection, the pharmaceutical compositions of the disclosure may be formulated in aqueous solutions, for example, in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art. In the methods described herein, an appropriate dosage level may generally be about 0.01 to 500 mg per kg patient body weight per day, which can be administered in single or multiple doses. The dosage level can be about 0.01 to about 250 mg/kg per day, such as 0.01 to about 100 mg/kg per day, for example, 0.01 to about 10 mg/kg per day, such as 0.04 to about 5 mg/kg per day, or about 0.5 to about 100 mg/kg per day. A suitable dosage level may be also about 0.05 to 100 mg/kg per day, or about 0.1 to 50 mg/kg per day or 1.0 mg/kg per day. Within this range the dosage may be 0.05 to 0.5, 0.5 to 5 or 5 to 50 mg/kg per day for example. The compounds may be administered on a regimen of 1 to 4 times per day, or once or twice per day or continuously per day or during a portion of the day. There may be a period of no administration followed by another regimen of administration. Administration of the compounds may be closely associated with the schedule of a second agent of administration.

It will be understood, however, that the specific dose level and frequency of dosage for any particular patient may be varied and will depend upon a variety of factors including the age, body weight, general health, sex, diet, mode and time of administration, rate of excretion, drug combination, the severity of the particular condition, and the host undergoing therapy.

A “humanized antibody” refers to an antibody that comprises a donor antibody binding specificity, i.e., the CDR regions of a donor antibody, typically a mouse monoclonal antibody, grafted onto human framework sequences. A “humanized antibody” as used herein binds to the same epitope as the donor antibody and typically has at least 25% of the binding affinity. Methods to determine whether the antibody binds to the same epitope are well known in the art, see, e.g., Harlow & Lane, Using Antibodies, A Laboratory Manual, Cold Spring Harbor Laboratory Press, 1999, which discloses techniques to epitope mapping or alternatively, competition experiments, to determine whether an antibody binds to the same epitope as the donor antibody. A humanized antibody that comprises a novel framework region is provided in the invention.

A “VH” or “VL”/“VK” “region” or “framework” of the invention refers to the variable region of the heavy chain (VH) or the variable region of the light/kappa chain (VL/VK) amino acid sequence that has at least 70% identity, often, at least 75%, 80%, 85%, 90%, 95%, 96%, 97%, 98%, or 99% identity, to an amino acid sequence of HJ8.5, the murine anti-tau antibody. A “framework” of a VH or VL chain refers to the framework regions of the chain not including the CDRs. The term as applied to each chain encompasses all of the framework regions.

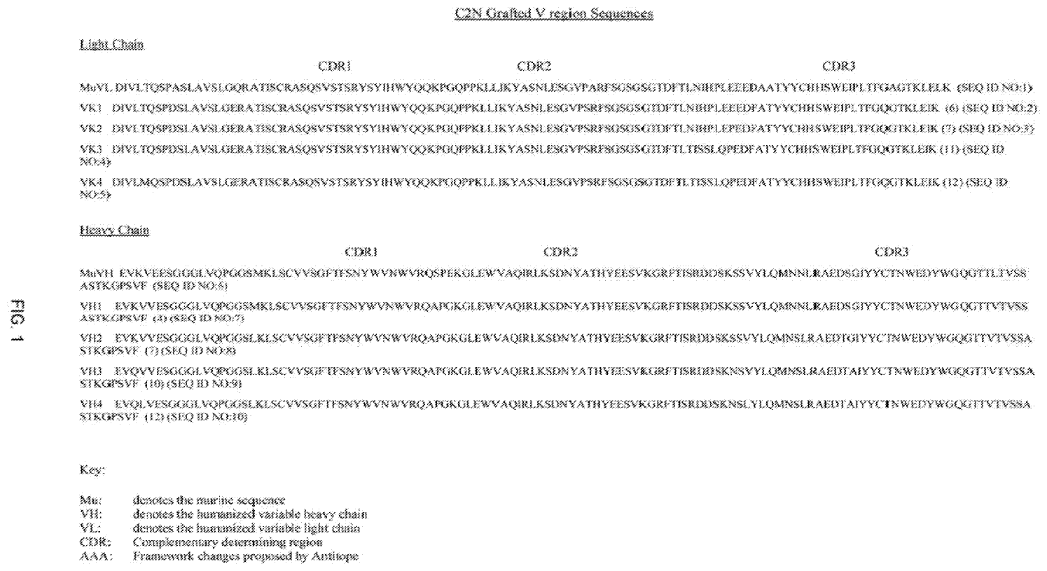

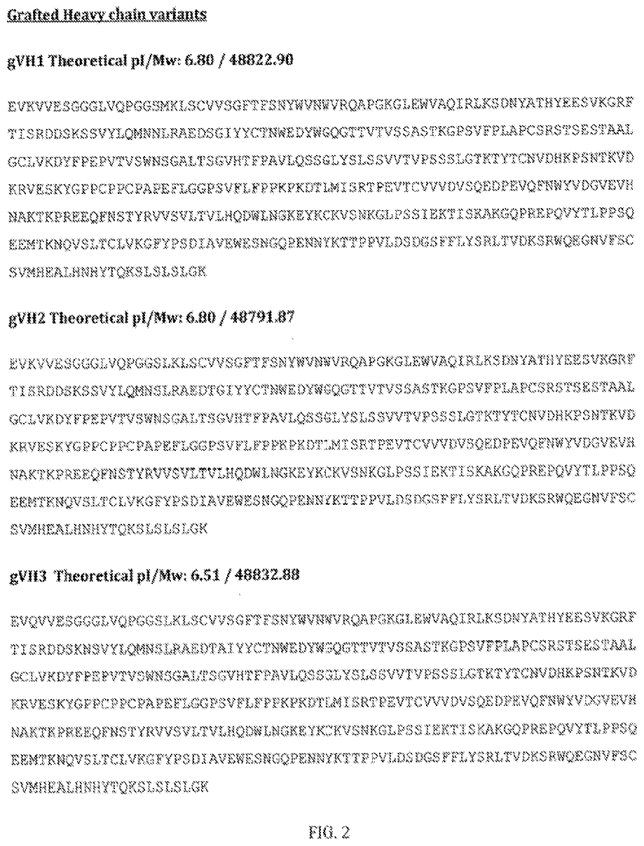

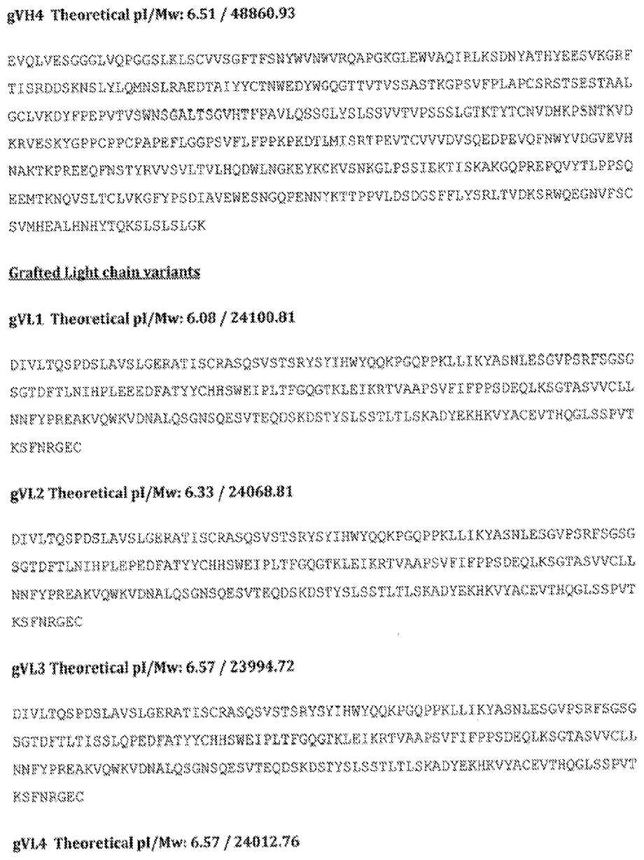

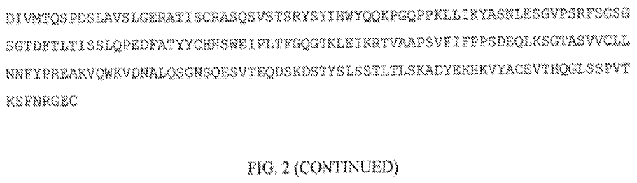

A “humanized anti-tau” or “anti-tau antibody” refers to a humanized antibody comprising a human framework sequence that has the binding specificity of the murine HJ8.5 grafted to that framework. A CDR of a humanized anti-tau antibody of the invention has at least 85%, more typically at least 90%, 95%, 96%, 97%, 98%, or 99% identity to a CDR of the heavy and light chain sequences in FIGS. 1 and 2. The amino acid sequence of the CDRs of the VH and VL/K regions are set forth in FIGS. 1 and 2.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. This linker can be between 5 and 30 amino acids long and usually consist of repeats of “GGGGS” amino acid sequence.

“F(ab′)2,” “Fab,” “Fab′” and “Fv” are antigen-binding fragments that can be generated from the variable region of IgG and IgM. These antigen-binding fragments vary in size (MW), valency and Fc content.

The phrase “fragment antigen-binding” or “(Fab fragment)” refers to a region on an antibody that binds to antigens. It is composed of one constant and one variable domain of each of the heavy and the light chain. These domains shape the paratope—the antigen-binding site—at the amino terminal end of the monomer. The two variable domains bind the epitope on their specific antigens.

“F(ab′)2 fragment” refers to a fragment that contains two antigen-binding regions joined at the hinge through disulfides. This fragment is void of most, but not all, of the Fc region.

“Fab′ fragments” can be formed by the reduction of F(ab′)2 fragments. The Fab′ fragment contains a free sulfhydryl group that may be alkylated or utilized in conjugation with an enzyme, toxin or other protein of interest. Fab′ is derived from F(ab′)2; therefore, it may contain a small portion of Fc.

“Fab” is a monovalent fragment that is produced from IgG and IgM, consisting of the VH, CHI and VL, CL regions, linked by an intramolecular disulfide bond.

“Fv” is the smallest fragment produced from IgG and IgM that contains a complete antigen-binding site. Fv fragments have the same binding properties and similar three-dimensional binding characteristics as Fab.

“Fc fragments” contain the CH2 and CH3 region and part of the hinge region held together by one or more disulfides and noncovalent interactions. Fc fragments are generated entirely from the heavy chain constant region of an immunoglobulin.

In one aspect, the invention provides composite humanized anti-tau antibodies and stable formulations thereof. Composite human antibody technology generates humanized non-immunogenic antibodies by avoiding T cell epitopes (deimmunisation) in variable region (V region) sequences (EP2,388,871). Unlike other humanization technologies that use single human V region frameworks as ‘acceptors’ for complimentarity-determining regions (CDRs) from the starting antibody (typically murine), Composite Human Antibodies™ comprise multiple sequence segments (‘composites’) derived from V regions of unrelated human antibodies.

In one embodiment the murine CDR sequences are fused with the constant region sequences of human IgG. The sequences are then modified to create a humanized anti-tau antibody.

Sequence segments derived from databases of unrelated human V regions are selected after determining amino acids that are considered critical for antigen binding of the starting antibody. All selected sequence segments derived from human V region databases are filtered for the presence of potential T cell epitopes using Antitope's in silico tools. Composite Human Antibodies™ retain affinity and specificity better than standard humanized antibodies due to the close fit of human sequence segments with all sections of the starting antibody V regions. Composite Human Antibodies™ are depleted of T cell epitopes and therefore considered both humanized and deimmunised.

In one embodiment, the invention antibodies are prepared by identifying candidate residues in the framework region to be mutated at specific sites within T cell epitopes. Invention antibodies may exhibit altered binding affinity and/or altered immunogenicity as compared to donor antibodies. Suitable formulations of the antibodies are prepared by identification of analytical methods to detect degradants present in the formulation. Experiments were performed under stress conditions and samples analyzed to evaluate sensitivity of the methods to be used to evaluate formulations.

C2N-8E12 anti-tau antibody has high sensitivity to mechanical and thermal stress. Over 50 formulation variables (pH, ionic strength, buffer system and excipients) were evaluated based on thermofluorescence screening and 15 formulation candidates were identified for further stability assessment. The second phase of screening focused on optimizing the formulation with the maximum physical stability. These 15 formulations are given in Table 7.

Methods known in the art can be used to map T cell epitopes within a protein sequence. For example, EpiScreen™ (EP1989544, Antitope, UK) is used to map T cell epitopes within a protein sequence to determine potential for immunogenicity, which is based on the number and potency of T cell epitopes within a sequence. EpiScreen™ T cell epitope mapping typically uses CD8+ T cell depleted peripheral blood mononuclear cells (PBMCs) from between 20-50 HLA-typed donors (selected to represent the natural variation found in the general population). Typically, 15mer peptides with 12 amino acid overlaps spanning a protein sequence are analyzed in a large number of replicate cultures for in vitro CD4+ T cell stimulation by 3H TdR incorporation. CD4+ T cell stimulation is often detected in two or three adjacent and overlapping peptides since the core 9mer that binds the MHC class II binding groove will be present in more than one peptide sequence. After the accurate identification of peptides that stimulate CD4+ T cells in vitro, in silico technologies can be used to design epitope-depleted (deimmunized) variants by determining the precise location of core 9mer sequences and the location of key MHC class II anchor residues. The EpiScreen™ assay can also be used to assess T cell activation of a particular humanized antibody.

In one embodiment, a single chain Fv humanized antibody of the invention, e.g., humanized anti-tau antibody, may bind as a monomer. Other exemplary single chain antibodies may form diabodies, triabodies, and tetrabodies.

Further the humanized antibodies of the invention, e.g., humanized anti-tau antibody may also form one component of a “reconstituted” antibody or antibody fragment, e.g., a Fab, a Fab′ monomer, a F(ab)′2 dimer, or an whole immunoglobulin molecule. Thus, a humanized antibody of the present invention may further comprise a human Fc region.

### Example 1

### Analytical Methods to Detect Degradants

This example illustrates identification of analytical methods to detect degradants present in the formulation.

Methods, Results and Discussion

Experiments were performed under stress conditions and samples analyzed to evaluate sensitivity of the methods to be used to evaluate formulations. Table 1 shows analytical methods used to evaluate C2N-8E12 degradation pathways.

The Indicative Analytical Methods Rating refers to sensitivity of C2N-8E12 to a particular stress condition, with a “high” rating indicating high sensitivity and a “Low” rating indicating low sensitivity. Some of these methods were used in Experiment 5.

TABLE 1
List of analytical methods to assess C2N-8E12 degradation pathways.

| | | | Indicative |
|---|---|---|---|
| | | | analytical |
| Sample | Degradation | | methods |
| No. | pathway | Analytical method | rating |
| 1 | Aggregation | SE-HPLC, T580/visual inspection, | High |
| | | MFI, non-reducing SDS-PAGE | |
| 2 | Degradation | SE-HPLC, T580/visual inspection, | High |
| | | MFI, non-reducing SDS-PAGE | |
| 3 | Deamidation | SE-HPLC, IEF | Low |
| 4 | Oxidation | SE-HPLC, T580/visual inspection, | Low |
| | | MFI | |
| 5 | Hydrolysis | SE-HPLC, SDS-PAGEs, IEF | High |

### Example 2

### Thermofluorescence Screening

This example illustrates the determination of thermal transition points of C2N-8E12.

The determination of thermal transition points of C2N-8E12 was carried out via thermofluorescence (TF) assays. The fluorescent dye binds to hydrophobic patches of the protein. During temperature increase, the protein unfolds and more dye can bind which results in an increase in the fluorescent signal caused by reduced water quenching. Therefore, higher thermal transition points (melting temperatures, Tₘ) indicate more stable conditions for C2N-8E12.

Using thermofluorescence assays, the thermal transition points of C2N-8E12 were determined for the starting materials with respect to different buffer properties. Namely pH, buffer system, ionic strength and excipients to adjust to physiological osmolality. A higher temperature indicates that the buffer system provided greater thermal stability to the antibody.

TABLE 2
TF results (in ° C.) of buffer screening round 1.

| | Concentration | | | | | | | |
|---|---|---|---|---|---|---|---|---|
| Buffer | buffer | Excipients | pH 7.5 | pH 7.0 | pH 6.5 | pH 6.0 | pH 5.5 | pH 5.0 |
| L- | 50 mM | 9% Saccharose | N/A | 66.9 | 65.6 | 59.8 | 59.4 | 55.4 |
| Histidine/ | 30 mM | | | 66.7 | 65.9 | 59.7 | 59.4 | 57.8 |
| HCl | 10 mM | | | 66.8 | 66.6 | 59.9 | 59.4 | 59.5 |
| | 50 mM | 5% Sorbitol | N/A | 66.7 | 66.5 | 59.7 | 59.4 | 56.7 |
| | 30 mM | | | 66.7 | 66.3 | 59.7 | 59.4 | 57.7 |
| | 10 mM | | | 66.8 | 66.6 | 59.8 | 59.4 | 59.6 |
| | 50 mM | 2% Glycine | N/A | 66.5 | 66.2 | 59.8 | 59.4 | 59.5 |
| | 30 mM | | | 66.6 | 66.2 | 59.8 | 59.4 | 59.5 |
| | 10 mM | | | 66.6 | 64.7 | 63.5 | 59.7 | 59.4 |
| 50 mM | 50 mM | 6.5% Saccharose | N/A | 65.7 | 64.8 | 59.7 | 59.2 | 56.5 |
| L- | | 3.5% Sorbitol | | 65.9 | 64.5 | 59.6 | 59.3 | 56.2 |
| Histidine/ | | 1.5% Glycine | | 65.8 | 65.0 | 59.8 | 59.3 | 57.5 |
| 25 mM | | | | | | | | |
| NaCl | | | | | | | | |
| 50 mM | 50 mM | 5.0% Saccharose | N/A | 65.0 | 64.4 | 59.4 | 59.3 | 56.1 |
| L- | | 2.5% Sorbitol | | 65.1 | 64.2 | 59.4 | 59.4 | 54.7 |
| Histidine/ | | 1.0% Glycine | | 65.3 | 64.9 | 59.5 | 59.3 | 57.9 |
| 50 mM | | | | | | | | |
| NaCl | | | | | | | | |
| 50 mM | 50 mM | 1.5% Saccharose | N/A | 66.0 | 65.1 | 59.6 | 59.3 | 55.3 |
| L- | | 1.0% Sorbitol | | 65.2 | 64.9 | 59.6 | 59.3 | 55.2 |
| Histidine/ | | 1.0% Glycine | | 65.1 | 65.4 | 59.6 | 59.4 | 55.6 |
| 100 mM | | | | | | | | |
| NaCl | | | | | | | | |

| | | | | | |
|---|---|---|---|---|---|
| Na- | 50 mM | 9% Saccharose | N/A | 59.8 | 59.6 |
| citrate | 30 mM | | | 59.7 | 59.6 |
| | 10 mM | | | 59.6 | 59.5 |
| | 50 mM | 5% Sorbitol | N/A | 59.7 | 59.5 |
| | 30 mM | | | 59.7 | 59.4 |
| | 10 mM | | | 59.7 | 59.3 |
| | 50 mM | 2% Glycine | N/A | 59.7 | 59.4 |
| | 30 mM | | | 59.6 | 59.3 |
| | 10 mM | | | 59.8 | 59.3 |

TABLE 3
TF results of buffer screening round 1.

| Buffer | Concentration buffer | Excipients | pH 7.5 | pH 7.0 | pH 6.5 | pH 6.0 | pH 5.5 | pH 5.0 |
|---|---|---|---|---|---|---|---|---|
| Na-acetate | 50 mM | 9% Saccharose | N/A | | | 66.3 | 59.9 | 59.4 |
| | 30 mM | | | | | 66.3 | 59.7 | 59.4 |
| | 10 mM | | | | | 66.5 | 59.8 | 59.5 |
| | 50 mM | 5% Sorbitol | N/A | | | 65.9 | 59.6 | 59.4 |
| | 30 mM | | | | | 65.8 | 59.7 | 59.3 |
| | 10 mM | | | | | 65.9 | 59.9 | 59.4 |
| | 50 mM | 2% Glycine | N/A | | | 65.7 | 59.9 | 59.4 |
| | 30 mM | | | | | 65.6 | 59.9 | 59.4 |
| | 10 mM | | | | | 65.5 | 59.8 | 59.8 |
| Na-Phosphate | 50 mM | 9% Saccharose | 64.2 | 64.5 | 65.1 | 65.1 | 59.6 | N/A |
| | 30 mM | | 64.1 | 64.7 | 65.5 | 65.4 | 59.6 | |
| | 10 mM | | 64.2 | 65.1 | 65.6 | 65.4 | 59.6 | |
| | 50 mM | 5% Sorbitol | 64.4 | 65.1 | 65.4 | 65.4 | 59.6 | N/A |
| | 30 mM | | 64.6 | 65.3 | 65.8 | 66.0 | 59.7 | |
| | 10 mM | | 64.8 | 65.7 | 66.3 | 66.3 | 59.7 | |
| | 50 mM | 2% Glycine | 65.7 | 65.8 | 65.8 | 65.6 | 59.8 | N/A |
| | 30 mM | | 65.7 | 65.8 | 66.1 | 65.7 | 59.7 | |
| | 10 mM | | 66.2 | 66.3 | 66.1 | 65.4 | 59.8 | |

| | | | | | |
|---|---|---|---|---|---|
| Glycyl-Glycine | 50 mM | 9% Saccharose | 66.6 | 65.9 | N/A |
| | 30 mM | | 66.2 | 65.9 | |
| | 10 mM | | 66.1 | 65.7 | |
| | 50 mM | 5% Sorbitol | 66.3 | 66.2 | N/A |
| | 30 mM | | 66.3 | 66.2 | |
| | 10 mM | | 66.3 | 65.9 | |
| | 50 mM | 2% Glycine | 66.3 | 66.3 | N/A |
| | 30 mM | | 66.2 | 66.0 | |
| | 10 mM | | 66.3 | 65.6 | |

TABLE 4
TF results of buffer screening round 2.

| | Concentration | | | | | |
|---|---|---|---|---|---|---|
| Buffer | buffer | Excipients | pH 7.5 | pH 7.0 | pH 6.5 | pH 6.0 |
| L- | 50 mM | 9% Saccharose | N/A | 66.9 | 66.2 | N/A |
| Histidine/HCl | 30 mM | | | 66.7 | 66.2 | |
| | 10 mM | | | 66.7 | 66.1 | |
| | 50 mM | 5% Sorbitol | N/A | 66.8 | 65.7 | |
| | 30 mM | | | 66.6 | 65.7 | |
| | 10 mM | | | 66.5 | 66.1 | |
| | 50 mM | 2% Glycine | N/A | 66.4 | 65.3 | |
| | 30 mM | | | 66.3 | 65.9 | |
| | 10 mM | | | 66.3 | 65.9 | |
| 50 mM L- | 50 mM | 6.5% Saccharose | N/A | 65.9 | 64.7 | |
| Histidine/25 mM | | 3.5% Sorbitol | | 65.8 | 64.6 | |
| NaCl | | 1.5% Glycine | | 65.7 | 64.7 | |
| 50 mM L- | 50 mM | 5.0% Saccharose | N/A | 65.7 | 64.5 | |
| Histidine/50 mM | | 2.5% Sorbitol | | 65.5 | 64.4 | |
| NaCl | | 1.0% Glycine | | 65.3 | 64.3 | |
| 50 mM L- | 50 mM | 1.5% Saccharose | N/A | 64.8 | 63.4 | |
| Histidine/100 mM | | 1.0% Sorbitol | | 64.5 | 63.9 | |
| NaCl | | 1.0% Glycine | | 64.7 | 64.0 | |
| Na-acetate | 50 mM | 9% Saccharose | N/A | | | 66.3 |
| | 30 mM | | | | | 66.4 |
| | 10 mM | | | | | 66.0 |
| | 50 mM | 5% Sorbitol | N/A | | | 66.5 |
| | 30 mM | | | | | 66.0 |
| | 10 mM | | | | | 65.8 |
| | 50 mM | 2% Glycine | N/A | | | 65.7 |
| | 30 mM | | | | | 65.8 |
| | 10 mM | | | | | 65.7 |
| Na-Phosphate | 30 mM | 9% Saccharose | N/A | 64.9 | 65.8 | 66.1 |
| | 10 mM | | | 65.2 | 65.9 | 65.6 |
| | 30 mM | 5% Sorbitol | N/A | 65.0 | 65.7 | 65.3 |
| | 10 mM | | | 65.1 | 65.9 | 65.7 |
| | 50 mM | 2% Glycine | 65.5 | 65.7 | 65.7 | 65.4 |
| | 30 mM | | 65.8 | 65.7 | 66.1 | 65.6 |
| | 10 mM | | 66.2 | 66.1 | 66.1 | 65.3 |

TABLE 5
TF results of buffer screening round 2.

| | Concentration | | pH | pH | pH | pH |
|---|---|---|---|---|---|---|
| Buffer | buffer | Excipients | 7.5 | 7.0 | 6.5 | 6.0 |

| | | | | | |
|---|---|---|---|---|---|
| Glycyl- | 50 mM | 9% | 66.9 | 66.3 | N/A |
| Glycine | 30 mM | Saccharose | 66.4 | 66.5 | |
| | 10 mM | | 66.4 | 66.3 | |
| | 50 mM | 5% Sorbitol | 66.1 | 66.4 | N/A |
| | 30 mM | | 66.5 | 66.1 | |
| | 10 mM | | 66.3 | 66.6 | |
| | 50 mM | 2% Glycine | 66.4 | 66.0 | N/A |
| | 30 mM | | 66.1 | 65.9 | |
| | 10 mM | | 66.0 | 65.3 | |

TABLE 6
TF results of buffer screening round 3.

| Buffer | Concentration buffer | Excipients | pH 7.5 | pH 7.0 | pH 6.5 | pH 6.0 |
|---|---|---|---|---|---|---|
| L-Histidine/HCl | 50 mM | 9% Saccharose | N/A | 66.9 | 65.9 | N/A |
| | 10 mM | | | 66.8 | N/A | |
| | 50 mM | 5% Sorbitol | N/A | 66.6 | N/A | |
| | 30 mM | | | 66.5 | | |
| | 10 mM | | | 66.5 | 66.3 | |
| | 50 mM | 2% Glycine | N/A | N/A | | 60.4 |
| | 30 mM | | | 66.3 | N/A | |
| 50 mM L- | 50 mM | 6.5% Saccharose | N/A | 65.7 | N/A | |
| Histidine/25 mM | | 3.5% Sorbitol | | 65.7 | | |
| NaCl | | | | | | |
| Na-acetate | 50 mM | 9% Saccharose | N/A | | | 66.2 |
| | 30 mM | | | | | 65.5 |
| | 50 mM | 5% Sorbitol | N/A | | | 65.7 |
| Na-Phosphate | 30 mM | 9% Saccharose | N/A | N/A | 66.0 | 66.1 |
| | 10 mM | | | 65.5 | 66.2 | N/A |
| | 10 mM | 5% Sorbitol | N/A | 66.2 | 66.1 | N/A |
| | 30 mM | 2% Glycine | N/A | N/A | 66.0 | N/A |
| | 10 mM | | 65.9 | 66.2 | 66.2 | |

| | | | | | |
|---|---|---|---|---|---|
| Glycyl-Glycine | 50 mM | 9% Saccharose | 66.4 | N/A | N/A |
| | 30 mM | | N/A | 66.3 | |
| | 30 mM | 5% Sorbitol | 66.3 | N/A | N/A |
| | 10 mM | | N/A | 66.2 | |
| | 50 mM | 2% Glycine | N/A | 66.0 | N/A |
| | 30 mM | | | 65.2 | |
| | 10 mM | | | 65.4 | |

Thermofluorescence investigations showed thermal transition points between 54.7° C. and 67.9° C. The highest Tₘ values were found in the range of pH 6.5-7.5 in histidine buffers with a trending of increasing stability from high to low ionic strengths. A Tₘ value of at least about 66° C. to 67° C., at pH 6-7, was considered optimal critical for a stable composition of C2N-8E12. The addition of saccharose and sorbitol as excipients for adjustment of physiological osmolality resulted in an increase in Tₘs.

### Example 3

### Forced Degradation Stability Study: Basis Buffer and Excipients

This example shows the results of a forced degradation study of various formulation candidates.

Based on thermofluorescence screenings in Example 2, 15 formulation candidates (FCs) were chosen and subjected to forced degradation. In Table 7, favourable FCs are shown in bold font, (+) shows significant signal differences, (o) shows non-significant signal differences and (−) shows no signal changes. FCs selected for the short term stability study in Example 4 were FC3, FC5 and FC9 in Table 7.

TABLE 7
Analytical results of basis buffer with excipients stability study.

| | | | | | | MFI | | | | |
|---|---|---|---|---|---|---|---|---|---|---|
| | | | SE-HPLC | | | Subvisible | Mean | | SDS-PAGE | |
| | Buffer | Visual | Aggregates | Monomer | Fragments | particles | ECD | | Non | |
| FC | composition | inspection | [Area-%] | [Area-%] | [Area-%] | [#/mL] | [μm] | IEF | reducing | Reducing |
| — | Reference | clear | 2.0 | 98.0 | <0.1 | 62,723 | 6.08 | − | − | − |
| | 5 ± 3° C. | solution with | | | | | | | | |
| | (10 mM L- | slight | | | | | | | | |
| | Histidine/ | precipitation | | | | | | | | |
| | HCl, 5.3% | | | | | | | | | |
| | Sorbitol pH | | | | | | | | | |
| | 6.5) | | | | | | | | | |
| 1 | 50 mM L- | clear | 2.2 | 97.6 | 0.1 | 541,756 | 3.31 | + | ∘ | ∘ |
| | Histidine/HCl, | solution | | | | | | | | |
| | 8.6% | with slight | | | | | | | | |
| | Saccharose | precipitation | | | | | | | | |
| | pH 7.0 | | | | | | | | | |
| 2 | 50 mM L- | slight turbid | 2.2 | 97.7 | <0.1 | 664,499 | 6.89 | ∘ | ∘ | − |
| | Histidine/ | solution with | | | | | | | | |
| | HCl, 8.6% | slight | | | | | | | | |
| | Saccharose | precipitation | | | | | | | | |
| | pH 6.5 | | | | | | | | | |
| 3 | 50 mM L- | clear | 2.1 | 97.7 | 0.2 | 461,727 | 7.28 | − | ∘ | ∘ |
| | Histidine/HCl, | solution | | | | | | | | |
| | 8.6% | with slight | | | | | | | | |
| | Saccharose | precipitation | | | | | | | | |
| | pH 6.0 | | | | | | | | | |
| 4 | 10 mM L- | slight turbid | 3.0 | 96.8 | 0.1 | 1,689,242 | 4.68 | ∘ | ∘ | ∘ |
| | Histidine/ | solution with | | | | | | | | |
| | HCl, 9.9% | precipitation | | | | | | | | |
| | Saccharose | | | | | | | | | |
| | pH 7.0 | | | | | | | | | |
| 5 | 50 mM L- | slight turbid | 2.3 | 97.5 | 0.2 | 1,228,986 | 3.39 | ∘ | ∘ | ∘ |
| | Histidine/ | solution with | | | | | | | | |
| | HCl, 5.3% | slight | | | | | | | | |
| | Sorbitol pH | precipitation | | | | | | | | |
| | 7.0 | | | | | | | | | |
| 6 | 10 mM L- | turbid | 2.4 | 97.4 | 0.2 | 1,577,900 | 13.43 | ∘ | ∘ | − |
| | Histidine/ | solution | | | | | | | | |
| | HCl, 5.3% | | | | | | | | | |
| | Sorbitol pH | | | | | | | | | |
| | 6.5 | | | | | | | | | |
| 7 | 30 mM L- | turbid | 2.1 | 97.6 | 0.2 | 12,691,483 | 5.06 | ∘ | + | ∘ |
| | Histidine/ | solution | | | | | | | | |
| | HCl, 1.9% | | | | | | | | | |
| | Glycine pH | | | | | | | | | |
| | 7.0 | | | | | | | | | |
| 8 | 30 mM Na- | clear | 3.0 | 96.9 | 0.1 | 353,304 | 8.25 | − | ∘ | − |
| | citrate, | solution | | | | | | | | |
| | 9.2% | with slight | | | | | | | | |
| | Saccharose | precipitation | | | | | | | | |
| | pH 6.0 | | | | | | | | | |
| 9 | 30 mM Na- | clear | 3.0 | 96.9 | <0.1 | 364,621 | 3.26 | ∘ | ∘ | ∘ |
| | acetate, | solution | | | | | | | | |
| | 9.2% | with slight | | | | | | | | |
| | Saccharose | precipitation | | | | | | | | |
| | pH 5.5 | | | | | | | | | |
| 10 | 30 mM Na- | clear | 2.9 | 97.0 | 0.1 | 388,431 | 2.86 | ∘ | ∘ | ∘ |
| | acetate, | solution with | | | | | | | | |
| | 2.0% Glycine | slight | | | | | | | | |
| | pH 5.5 | precipitation | | | | | | | | |
| 11 | 10 mM Na- | turbid | 3.9 | 96.0 | 0.1 | 842,968 | 9.07 | ∘ | ∘ | ∘ |
| | phosphate, | solution | | | | | | | | |
| | 9.9% | | | | | | | | | |
| | Saccharose | | | | | | | | | |
| | pH 6.5 | | | | | | | | | |
| 12 | 10 mM Na- | clear | 5.0 | 94.8 | 0.2 | 101,476 | 10.69 | + | ∘ | ∘ |
| | phosphate, | solution | | | | | | | | |
| | 5.3% Sorbitol | with slight | | | | | | | | |
| | pH 7.0 | precipitation | | | | | | | | |
| 13 | 10 mM Na- | turbid | 3.3 | 96.4 | 0.3 | 2,511,213 | 16.13 | + | ∘ | − |
| | phosphate, | solution | | | | | | | | |
| | 2.2% Glycine | | | | | | | | | |
| | pH 6.5 | | | | | | | | | |
| 14 | 50 mM | clear | 2.7 | 97.1 | 0.2 | 1,053,919 | 2.13 | + | ∘ | ∘ |
| | Glycyl- | solution | | | | | | | | |
| | Glycine, | with slight | | | | | | | | |
| | 8.6% | precipitation | | | | | | | | |
| | Saccharose | | | | | | | | | |
| | pH 7.5 | | | | | | | | | |
| 15 | 30 mM | clear | 2.6 | 97.3 | 0.1 | 5,198,576 | 1.84 | ∘ | ∘ | − |
| | Glycyl- | solution | | | | | | | | |
| | Glycine, | with slight | | | | | | | | |
| | 9.2% | precipitation | | | | | | | | |
| | Saccharose | | | | | | | | | |
| | pH 7.0 | | | | | | | | | |

Aggregate levels of no more than 5%, and/or monomer levels of at least 95% are considered optimal for a stable composition of C2N-8E12.

TABLE 8
Analytical results of basis buffer with excipients stability study, favourable
FCs are in bold font.

| | | | | | | MFI | |
|---|---|---|---|---|---|---|---|
| | | | SE-HPLC | | | Subvisible | Mean |
| | | | Aggregates | Monomer | Aggregates | particles [#/ | ECD |
| FC | Buffer composition | Visual inspection | [Area-%] | [Area-%] | [Area-%] | mL] | [μm] |
| — | Reference 5 ± 3° C. | clear solution with | 2.2 | 97.8 | <0.1 | 37,311 | 6.34 |
| | (10 mM L-Histidine/HCl, | slight precipitation | | | | | |
| | 5.3% Sorbitol pH 6.5) | | | | | | |
| 1 | 50 mM L-Histidine/HCl, | clear solution | 2.0 | 98.0 | <0.1 | 445,022 | 2.33 |
| | 8.6% Saccharose pH 7.0 | | | | | | |
| 2 | 50 mM L-Histidine/HCl, | clear solution with | 1.8 | 98.1 | <0.1 | 283,320 | 4.37 |
| | 8.6% Saccharose pH 6.5 | slight precipitation | | | | | |
| 3 | 50 mM L-Histidine/HCl, | clear solution | 1.8 | 98.2 | <0.1 | 394,124 | 3.44 |
| | 8.6% Saccharose pH 6.0 | | | | | | |
| 4 | 50 mM L-Histidine/HCl, | clear solution | 1.9 | 98.0 | <0.1 | 1,019,868 | 2.57 |
| | 5.3% Sorbitol pH 7.0 | | | | | | |
| 5 | 30 mM Na-citrate, 9.2% | clear solution | 2.3 | 97.7 | <0.1 | 320,798 | 5.07 |
| | Saccharose pH 6.0 | | | | | | |
| 6 | 30 mM Na-acetate, | clear solution | 2.1 | 97.9 | <0.1 | 539,049 | 2.01 |
| | 9.2% Saccharose pH 5.5 | | | | | | |
| 7 | 30 mM Na-acetate, | clear solution | 6.1 | 93.8 | <0.1 | 481,521 | 2.02 |
| | 2.0% Glycine pH 5.5 | | | | | | |
| 8 | 30 mM Glycyl-Glycine, | clear solution | 2.1 | 97.9 | <0.1 | 646,863 | 2.56 |
| | 9.2% Saccharose pH 7.0 | | | | | | |

### Example 4

### Stability Study: Addition of Surfactants

FCs (formulation candidates) with moderate performance in the buffer and excipients stability study (e.g., aggregate level, particle size) were selected to be able to detect potential beneficial effects of the surfactants on product stability. The study was performed in a full factorial Design of Experiments (DoE) approach with three surfactants to be tested in a specific concentration range. The results of visual inspection, particle number and size suggests a trend of negative impact trend upon Tween20 addition.

Surfactant combinations with center point conditions for FC 02 from Table 9 showed a slight reduction in aggregate levels. For FC 09 from Table 9, a clear decrease in visual precipitation, particle number and size was observed when a surfactant combination was added. Therefore, it cannot be excluded that surfactant effects are dependent on the basis buffer and/or excipients.

TABLE 9
Results of surfactant stability study.

| | | SE-HPLC | | | MFI | |
|---|---|---|---|---|---|---|
| Buffer and surfactant | | Aggregates | Monomer | Fragments | [Particles/ | ECD |
| composition | Visual inspection | [%] | [%] | [%] | mL] | [μm] |
| FC 08 unstressed | clear solution with | 2.1 | 97.9 | <0.1 | 33,192 | 6.36 |
| | slight precipitation | | | | | |
| FC 08 stressed | clear solution with | 2.7 | 97.2 | 0.1 | 104,559 | 6.13 |
| | slight precipitation | | | | | |
| FC 08, 0.02% Tween 20 | turbid solution | 2.7 | 97.2 | 0.2 | 484,860 | 9.95 |
| FC 08, 0.70% HPβCD | clear solution with | 2.7 | 97.2 | 0.1 | 79,113 | 4.5 |
| | precipitation | | | | | |
| FC 08, 40 μM EDTA | clear solution with | 2.6 | 97.2 | 0.1 | 76,920 | 7.21 |
| | slight precipitation | | | | | |
| FC 08, 0.02% Tween 20, | clear solution with | 2.7 | 97.2 | 0.1 | 51,166 | 6.66 |
| 0.70% HPβCD | slight precipitation | | | | | |
| FC 08, 0.02% Tween 20, | slight turbid solution | 2.6 | 97.3 | 0.2 | 143,383 | 9.99 |
| 40 μM EDTA | with slight | | | | | |
| | precipitation | | | | | |
| FC 08, 0.70% HPβCD, | clear solution with | 2.6 | 97.2 | 0.2 | 47,995 | 7.34 |
| 40 μM EDTA | slight precipitation | | | | | |
| FC 08, 0.02% Tween 20, | clear solution with | 2.6 | 97.3 | 0.2 | 70,005 | 7.66 |
| 0.70% HPPCD, 40 μM | slight precipitation | | | | | |
| EDTA | | | | | | |
| FC 08, 0.01% Tween 20, | turbid solution | 2.6 | 97.3 | 0.2 | 360,301 | 10.23 |
| 0.35% HPβCD, 20 μM | (slightly opalescent) | | | | | |
| EDTA | | | | | | |
| FC 08, 0.01% Tween 20, | turbid solution | 2.6 | 97.3 | 0.2 | 495,810 | 8.89 |
| 0.35% HPβCD, 20 μM | (slightly opalescent) | | | | | |
| EDTA | | | | | | |
| FC 08, 0.01% Tween 20, | turbid solution | 2.6 | 97.3 | 0.2 | 565,876 | 7.6 |
| 0.35% HPβCD, 20 μM | (slightly opalescent) | | | | | |
| EDTA | | | | | | |
| FC 02 stressed | turbid solution | 2.3 | 97.5 | 0.2 | 1,221,431 | 7.18 |
| FC 02, 0.01% Tween 20, | turbid solution | 2.1 | 97.6 | 0.3 | 1,590,704 | 9.1 |
| 0.35% HPβCD, 20 μM | | | | | | |
| EDTA | | | | | | |
| FC 09 stressed | clear solution with a | 2.6 | 97.2 | 0.2 | 329,642 | 6.44 |
| | lot of large | | | | | |
| | precipitations | | | | | |
| FC 09, 0.01% Tween 20, | clear solution with | 2.5 | 97.4 | 0.2 | 32,576 | 2.77 |
| 0.35% HPβCD, 20 μM | slight precipitation | | | | | |
| EDTA | | | | | | |

FC02: 50 mM L-Histidine/HCl, 8.6% Saccharose pH 6.5;
FC08: 30 mM Na-citrate, 9.2% Saccharose pH 6.0,
FC09: 30 mM Na-acetate, 9.2% Saccharose pH 5.5

### Example 5

### Selection of Formulation Candidates for Short Term Stability Study

The following five buffer systems were selected for the short term stability study with the rationale indicated in Table 10.

TABLE 10
Final formulation candidates for three-month short term stability study.

| No | Buffer system | Rationale for selection |
|---|---|---|
| 1 | 50 mM L-Histidine/HCl, 8.6% | Overall performance of FC3 in Table 7 in the buffer and |
| | Saccharose pH 6.0 | excipient screening |
| 2 | 50 mM L-Histidine/HCl, 8.6% | Selection based on good experience with |
| | Saccharose pH 6.0, 0.02% Tween | histidine/Tween20 combination even if specific |
| | 20 | composition was not covered in the surfactant screening |
| 3 | 50 mM L-Histidine/HCl, 5.3% | Good performance after histidine/saccharose combination |
| | Sorbitol | in basis buffer & excipients stability study |
| | pH 7.0 | |
| 4 | 30 mM Na-acetate, 9.2% | To evaluate HPβCD alone. Showed beneficial effect in |
| | Saccharose pH 5.5, 0.7% HPβCD | combination with other surfactants and combining 3 |
| | | surfactants |
| 5 | 30 mM Na-acetate, 9.2% | Significant beneficial effect observed in surfactant |
| | Saccharose pH 5.5, 0.01% | screening (on particle number/size) |
| | Tween 20, 0.35% HPβCD, | |
| | 20 μM EDTA | |

The summarized results of the short term stability study after two month are listed in Table 11.

TABLE 11
Summary of results of short term stability study after
two month incubation period.

| Analytical method | Preferred formulation candidate (FC) |
|---|---|
| Visual inspection | FC2 = FC5 > FC1 > FC4 > FC3 |
| SE-HPLC | FC3 > FC2 = FC1 > FC5 > FC4 |
| MFI | FC5 > FC2 > FC1 > FC4 > FC3 |
| Non-reducing SDS-PAGE | FC2 = FC1 > FC4 > FC5 > FC3 |
| Reducing SDS-Page | FC2 = FC1 > FC4 = FC5 > FC3 |
| IEF | FC2 = FC4 = FC5 > FC1 > FC3 |

After 2 months incubation time, FC2 of Table 9 showed the overall best performance for all temperatures regarding stability of C2N-8E12. FC2 of Table 9, namely 20 g C2N-8E12/L, 50 mM Histidine, 8.6% Saccharose, 0.02% Tween 20, at pH 6.0, was selected as a leading formulation composition.

After three months incubation time at room temperature, the leading candidate FC2 of Table 9 was confirmed to have desired stability, as evidenced by purity >98% and <2% aggregates. Especially, a lower turbidity was achieved with the new formulation and the formation of particles after freeze/thaw was significantly reduced or avoided.

Where the amount of an ingredient is presented as a percentage, it refers to the percentage by weight of the ingredient in the composition based on the total weight of the composition, unless the context indicates otherwise.

Although the invention has been described with reference to the above examples, it will be understood that modifications and variations are encompassed within the spirit and scope of the invention. Accordingly, the invention is limited only by the following claims.

What is claimed is:

1. A composition comprising:
(i) a humanized antibody which specifically binds TAU comprising a heavy chain variable (VH) region and a light chain variable (VL) region; and
(ii) a buffering system that optionally contains an excipient and/or optionally contains a surfactant.

2. The composition of claim 1, wherein the humanized antibody is C2N-8E12.

3. The composition of claim 1, wherein the concentration of the humanized antibody in the buffering system is about 20 mg/mL.

4. The composition of claim 1, wherein the excipient is saccharose, sorbitol or glycine.

5. The composition of claim 1, wherein the surfactant is Tween20.

6. The composition of claim 1, wherein the buffering system is selected from the group consisting of: (a) 50 mM L-Histidine/HCl, 8.6% Saccharose at pH 7.0; (b) 50 mM L-Histidine/HCl, 8.6% Saccharose at pH 6.5; (c) 50 mM L-Histidine/HCl, 8.6% Saccharose at pH 6.0; (d) 50 mM L-Histidine/HCl, 5.3% Sorbitol at pH 7.0; (e) 30 mM Na-citrate, 9.2% Saccharose at pH 6.0; (f) 30 mM Na-acetate, 9.2% Saccharose at pH 5.5; and (g) 30 mM Na-acetate, 2.0% Glycine at pH 5.5; and (h) 30 mM Glycyl-Glycine, 9.2% Saccharose at pH 7.0.

7. The composition of claim 1, wherein the buffering system comprises 50 mM Histidine, and 8.6% Saccharose.

8. The composition of claim 7, wherein the buffering system further comprises a surfactant.

9. The composition of claim 8, wherein the surfactant is polysorbate 20.

10. The composition of claim 9, wherein polysorbate 20 is present in an amount of about 0.02%.

11. The composition of claim 10, wherein the concentration of the humanized antibody in the buffering system is about 20 mg/mL.

12. The composition of claim 1, wherein the buffering system comprises about 50 mM Histidine, about 8.6% Saccharose and about 0.02% Tween 20 at about pH 6.0.

13. A composition comprising:
(i) a humanized antibody which specifically binds TAU comprising a heavy chain variable (VH) region as set forth in FIGS. 1 and 2 and a light chain variable (VL) region as set forth in FIGS. 1 and 2; and
(ii) a buffering system comprising histidine, citrate, or glyclglycine, and that optionally contains an excipient and/or optionally contains a surfactant.

14. The composition of claim 13, wherein the buffering system comprises histidine and an excipient selected from saccharose, sorbitol, and combinations thereof.

15. The composition of claim 13, wherein the buffering system comprises citrate and an excipient selected from saccharose, glycine, and combinations thereof.

16. The composition of claim 13, wherein the buffering system comprises glycylglycine and the excipient is saccharose.

17. The composition of claim 13, wherein the buffering system comprises histidine/HCl and does not include sodium.

18. The composition of claim 13, wherein the anti-tau antibody is present at a concentration of about 20 mg/mL and the composition has a monomer content of about >95%, a pH between about 5 and 6, and is stable for at least three months when stored at about 5° C.

19. The composition of claim 13, wherein the buffering system comprises about 50 mM Histidine, about 8.6% Saccharose and about 0.02% Tween 20 at about pH 6.0.


SEQUENCE LISTING (18 sequences)

SEQ ID NO: 1 - protein, 442 aa, Homo sapiens
EVKVVESGGGLVQPGGSMKLSCVVSGFTFSNYWVNWVRQAPGKGLEWVAQIRLKSDNYATHYEESVKGRFTISRDDSKSSVYLQMNNLRAEDSGIYYCTNWEDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 442 aa, Homo sapiens
EVKVVESGGGLVQPGGSLKLSCVVSGFTFSNYWVNWVRQAPGKGLEWVAQIRLKSDNYATHYEESVKGRFTISRDDSKSSVYLQMNSLRAEDTGIYYCTNWEDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 442 aa, Homo sapiens
EVQVVESGGGLVQPGGSLKLSCVVSGFTFSNYWVNWVRQAPGKGLEWVAQIRLKSDNYATHYEESVKGRFTISRDDSKNSVYLQMNSLRAEDTAIYYCTNWEDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 442 aa, Homo sapiens
EVQLVESGGGLVQPGGSLKLSCVVSGFTFSNYWVNWVRQAPGKGLEWVAQIRLKSDNYATHYEESVKGRFTISRDDSKNSLYLQMNSLRAEDTAIYYCTNWEDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 218 aa, Homo sapiens
DIVLTQSPDSLAVSLGERATISCRASQSVSTSRYSYIHWYQQKPGQPPKLLIKYASNLESGVPSRFSGSGSGTDFTLNIHPLEEEDFATYYCHHSWEIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 218 aa, Homo sapiens
DIVLTQSPDSLAVSLGERATISCRASQSVSTSRYSYIHWYQQKPGQPPKLLIKYASNLESGVPSRFSGSGSGTDFTLNIHPLEPEDFATYYCHHSWEIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 218 aa, Homo sapiens
DIVLTQSPDSLAVSLGERATISCRASQSVSTSRYSYIHWYQQKPGQPPKLLIKYASNLESGVPSRFSGSGSGTDFTLTISSLQPEDFATYYCHHSWEIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 218 aa, Homo sapiens
DIVMTQSPDSLAVSLGERATISCRASQSVSTSRYSYIHWYQQKPGQPPKLLIKYASNLESGVPSRFSGSGSGTDFTLTISSLQPEDFATYYCHHSWEIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 111 aa, Homo sapiens
DIVLTQSPASLAVSLGQRATISCRASQSVSTSRYSYIHWYQQKPGQPPKLLIKYASNLESGVPARFSGSGSGTDFTLNIHPLEEEDAATYYCHHSWEIPLTFGAGTKLELK

SEQ ID NO: 10 - protein, 111 aa, Homo sapiens
DIVLTQSPDSLAVSLGERATISCRASQSVSTSRYSYIHWYQQKPGQPPKLLIKYASNLESGVPSRFSGSGSGTDFTLNIHPLEEEDFATYYCHHSWEIPLTFGQGTKLEIK

SEQ ID NO: 11 - protein, 111 aa, Homo sapiens
DIVLTQSPDSLAVSLGERATISCRASQSVSTSRYSYIHWYQQKPGQPPKLLIKYASNLESGVPSRFSGSGSGTDFTLNIHPLEPEDFATYYCHHSWEIPLTFGQGTKLEIK

SEQ ID NO: 12 - protein, 111 aa, Homo sapiens
DIVLTQSPDSLAVSLGERATISCRASQSVSTSRYSYIHWYQQKPGQPPKLLIKYASNLESGVPSRFSGSGSGTDFTLTISSLQPEDFATYYCHHSWEIPLTFGQGTKLEIK

SEQ ID NO: 13 - protein, 111 aa, Homo sapiens
DIVMTQSPDSLAVSLGERATISCRASQSVSTSRYSYIHWYQQKPGQPPKLLIKYASNLESGVPSRFSGSGSGTDFTLTISSLQPEDFATYYCHHSWEIPLTFGQGTKLEIK

SEQ ID NO: 14 - protein, 124 aa, Homo sapiens
EVKVEESGGGLVQPGGSMKLSCVVSGFTFSNYWVNWVRQSPEKGLEWVAQIRLKSDNYATHYEESVKGRFTISRDDSKSSVYLQMNNLRAEDSGIYYCTNWEDYWGQGTTLTVSSASTKGPSVF

SEQ ID NO: 15 - protein, 124 aa, Homo sapiens
EVKVVESGGGLVQPGGSMKLSCVVSGFTFSNYWVNWVRQAPGKGLEWVAQIRLKSDNYATHYEESVKGRFTISRDDSKSSVYLQMNNLRAEDSGIYYCTNWEDYWGQGTTVTVSSASTKGPSVF

SEQ ID NO: 16 - protein, 124 aa, Homo sapiens
EVKVVESGGGLVQPGGSLKLSCVVSGFTFSNYWVNWVRQAPGKGLEWVAQIRLKSDNYATHYEESVKGRFTISRDDSKSSVYLQMNSLRAEDTGIYYCTNWEDYWGQGTTVTVSSASTKGPSVF

SEQ ID NO: 17 - protein, 124 aa, Homo sapiens
EVQVVESGGGLVQPGGSLKLSCVVSGFTFSNYWVNWVRQAPGKGLEWVAQIRLKSDNYATHYEESVKGRFTISRDDSKNSVYLQMNSLRAEDTAIYYCTNWEDYWGQGTTVTVSSASTKGPSVF

SEQ ID NO: 18 - protein, 124 aa, Homo sapiens
EVQLVESGGGLVQPGGSLKLSCVVSGFTFSNYWVNWVRQAPGKGLEWVAQIRLKSDNYATHYEESVKGRFTISRDDSKNSLYLQMNSLRAEDTAIYYCTNWEDYWGQGTTVTVSSASTKGPSVF